(19) United States
(12) Patent Application Publication
(10) Pub. No.: US 2003/0013118 A1
(43) Pub. Date: Jan. 16, 2003

(54) METHODS OF DETERMINING RESISTANCE TO TREATMENT FOR HEPATITIS C VIRUS

(76) Inventors: ALBERT EDGE, CAMBRIDGE, MA (US); NEZAM H. AFDHAL, CHARLESTOWN, MA (US)

Correspondence Address: LAHIVE & COCKFIELD, 28 STATE STREET, BOSTON, MA, 02109, US

(21) Appl. No.: 09/406,997

(22) Filed: Sep. 28, 1999

Related U.S. Application Data
(60) Provisional application No. 60/102,323, filed on Sep. 28, 1998.

(51) Int. Cl. C12Q 1/70; G01N 33/53; C12P 21/06; A61K 39/29

(52) U.S. Cl. 435/7.1; 435/5; 435/69.1; 424/225.1

(57) ABSTRACT

Methods for identifying hepatitis C virus (HCV)-infected subjects responsive to treatment for HCV infection or unlikely to respond to treatment for HCV infection are described. The level of Th2 cytokines in the subject during treatment serves as an indicator of whether the subject is likely to respond to treatment for HCV, e.g., interferon treatment. An elevated level of at least one Th2 cytokine during treatment indicates that an HCV-infected subject is unlikely to respond to a treatment for HCV. A decreased level of at least one Th2 cytokine indicates that an HCV-infected subject is responsive to a treatment for HCV. In a preferred embodiment, IL-10 levels are detected to identify subjects responsive to or unlikely to respond to interferon treatment.

### BACKGROUND OF THE INVENTION

Hepatitis C virus (HCV) is the major cause of post-transfusion hepatitis. More than 50% of acutely infected individuals progress to a chronic carrier state that frequently results in cirrhosis. This is in contrast to hepatitis B virus (HBV) in which less than 10% of patients with acute infection will become chronic. Davis et al. (1994) Am. J. Med. 96 (suppl. 1A):41-44. In addition, HCV infection is an independent risk factor for development of hepatocellular carcinoma. Siato et al. (1990) Proc. Natl Acad. Sci. 87:6547-6549; Tsukuma et al. (1993) N. Engl. J. Med. 328(25):1797-1801.

An approved therapy for HCV infection is a 6- to 12-month course of interferon-α treatment. Interferons (IFN) are a family of cytokines which have potent antiviral activity. Type I Interferons, i.e., IFN-α and IFN-β, also exhibit immunomodulatory actions such as inducing expression of major histocompatibility complex (MHC) class I and class II proteins, inhibiting the production of IL-1 and stimulating natural killer cells. It has been postulated that IFN-α may contribute to the elimination of chronic HCV infection by enhancing the host immune response. Davis et al. (1994) Am. J. Med. 96 (suppl. 1A):41-44. In several studies, however, it has been shown that IFN-α treatment leads to sustained improvement in only about 20% of patients having chronic HCV. The remaining chronic HCV-infected patients treated with IFN-α either demonstrate no significant improvement in HCV-associated symptoms or initially demonstrate improvement but then relapse to pre-treatment symptoms. With the high probability that IFN-α treatment will not lead to sustained improvement in patients having HCV and in view of the associated side effects of IFN-α treatment (e.g., fatigue, depression, bone marrow suppression and/or autoimmune thyroid disease), the decision to proceed with IFN-α treatment can be difficult. Davis et al. (1994) Am. J. Med. 96 (suppl. 1A):41-44. In addition, other antiviral analogs used for treatment of HCV have been shown to have limited efficacy and may also be associated with considerable side effects. Di Bisceglie et al. (1992) Hepatology 16(3):649-654.

Several attempts have been made to identify pretreating factors that could identify HCV-infected patients most likely to respond to IFN-α treatment. Age, gender, body weight, source of infection, duration of disease, and the degree of serum alanine aminotransferase (ALT) elevation have all been evaluated and found to not be predictable for a response to IFN-α treatment. See Davis et al. (1990) Hepatology 12:905; Farrel et al. (1991) GastroenteroL Jpn. 26 (suppl. 3):243-246.

Recently, studies have focused on the immune mechanisms involved in the pathogenesis of chronic HCV and the immune mechanisms involved with treatment of HCV with interferon by determining whether patients with this infection, in the presence or absence of interferon treatment, exhibit an altered immune response to HCV. See, e.g., Kobayashi et al. (1998) J. Gastroenterol. 33:500-507; Andreone et al. (1997) Hepatology 26 (4 PART 2):410S. The nature of the immune response against infectious agents can be determined in part by the pattern of cytokines secreted by T lymphocytes (i.e., type 1 T helper cell (Th1) responses and/or type 2 T helper cell (Th2) responses). See, e.g., Kobayashi et al. (1998) J. Gastroenterol. 33:500-507; Andreone et al. (1997) Hepatology 26 (4 PART 2):410S; Schendene et al. (1996) J. Gastroenterol. 110 (4 Supp); Cacciarelli et al. (1996) Hepatology 24(1):6-9.

A need exists, however, for an approach to evaluate patients having HCV infection to identify those patients likely to respond to treatment for HCV infection.

### SUMMARY OF THE INVENTION

The present invention features methods for identifying hepatitis C virus (HCV)-infected subjects, e.g., chronic HCV-infected subjects, likely to respond to treatment for hepatitis C virus infection or unlikely to respond to treatment for hepatitis C virus infection. The methods include determining the level of at least one Th2 cytokine in a subject diagnosed with and/or being treated for HCV infection wherein an elevated level of at least one Th2 cytokine (e.g., measured in cells taken from the liver, measured in cells taken from the periphery, or measured in serum) indicates that the subject is unlikely to respond to treatment for HCV infection. In another embodiment, the invention features methods for identifying HCV-infected subjects responsive to treatment for HCV infection wherein a reduced level of at least one Th2 cytokine (e.g., measured in cells taken from the liver, measured in cells taken from the periphery, or measured in serum) indicates that the subject is responsive to treatment for HCV infection. Treatments for HCV infection include interferon treatment, such as type I interferon treatment. Examples of type I interferon treatments include interferon α and/or interferon β treatment.

In one embodiment of the invention, a biological sample, e.g., a blood or biopsy sample, is obtained from a subject diagnosed with and/or being treated for HCV infection and the level of at least one Th2 cytokine (e.g., measured in cells taken from the liver, measured in cells taken from the periphery, or measured in serum) is determined in the sample. An elevated level of at least one Th2 cytokine prior to and/or during treatment indicates that the subject is unlikely to respond to treatment for HCV infection; whereas a level of a Th2 cytokine similar to that observed in an uninfected individual (or a reduction in an above-normal level of at least one Th2 cytokine during treatment) indicates that the subject will be responsive to treatment for HCV infection.

Levels of Th2 cytokines that can be determined include IL-4, IL-5, IL-6 and/or IL-10. Preferably, peripheral or intrahepatic IL-10 levels are determined (e.g., measured in cells taken from the liver), wherein an elevated level of IL-10 indicates that the subject is unlikely to respond to interferon treatment and a level of IL-10 similar to that in an uninfected individual (or a decrease in an elevated level upon treatment) indicates that the subject is likely to respond to interferon treatment.

Other features and advantages of the invention will be apparent from the following detailed description and claims.

### DETAILED DESCRIPTION OF THE INVENTION

This invention features methods of identifying hepatitis C virus (HCV)-infected subjects that are likely to respond to treatment for hepatitis C virus (HCV) infection or that are unlikely to respond to treatment for HCV infection. The methods of the invention include determining the level of at least one Th2 cytokine in the subject having HCV infection during treatment, wherein a level of at least one Th2 cytokine higher than that seen in an uninfected control individual indicates that the subject is unlikely to respond to treatment for HCV infection. Alternatively, according to the methods of the invention, a level of at least one Th2 cytokine in an HCV-infected subject diagnosed with and/or being treated for HCV infection which is comparable to (e.g., the same as or lower than) that seen in an uninfected control individual (or which is lower that that seen in the same infected individual prior to treatment) indicates that the subject is likely to respond to treatment for HCV infection.

The methods of the invention are particularly useful for determining responsiveness or predicting resistance to treatment for HCV in human subjects having HCV. Hepatitis C virus (HCV) infection refers to a clinical disorder caused by infectious agents, e.g., viral agents, which are antigenically and genetically different from hepatitis A virus and hepatitis B virus. Symptoms of HCV infection include at least one or more of the following: fever, nausea, vomiting and jaundice. Infection may be associated with inflammation of the liver and/or necrosis of liver cells. In acute stages, HCV infection is generally milder than hepatitis B virus, but a greater proportion of HCV infections become chronic. As used herein, the term “acute HCV” refers to the initial onset of HCV. Symptoms of acute HCV include at least one or more of the following: malaise, jaundice, a rise in alanine aminotransferase (ALT) levels, the presence of HCV RNA, and the presence of anti-HCV antibodies. As symptoms of HCV persist in a subject, HCV can progress from acute HCV to a chronic state. “Chronic HCV”, as used herein, refers to the persistence of HCV infection in a subject. Patients can be classified as having chronic HCV based on the persistence of elevated serum ALT levels and/or the presence of serum HCV RNA and/or the presence of anti-HCV antibodies in a patient over a period of at least about 4 months, preferably at least about 6 months, more preferably 7, 8, 9, 10, 11, or 12 months.

Methods of detecting HCV infection and monitoring symptoms of HCV in a subject are known in the art. For example, radioimmunoassays can be used to determine the presence of anti-HCV antibodies in the sera of a subject. See, e.g., Kuo et al. (1989) Science 244:362-364. Alternatively, anti-hepatitis C virus antibody to HCV antigen can be detected by fluorescent antibody blocking (Alter et al. 1992. New England Journal of Medicien. 327:1899). In addition, several methods are known in the art for detecting HCV RNA in a biological sample. For example, nucleic acid probes (e.g., labeled nucleic acid probes) capable of hybridizing to HCV mRNA can be used to detect the presence of HCV mRNA, preferably serum HCV mRNA. Nucleotide sequences which encode the HCV genome are known in the art. See e.g., EP 0318216, EP 0388232, EP 398748; Kato et al. (1990) Proc. Natl Acad. Sci USA 87:9524-9528; Choo et al. (1990) Proc. Natl Acad Sci. USA 88:2451-2455; and Choo et al. (1989) Science 244:359-362. Thus, using, for example, the nucleotide sequence of cDNA encoding HCV genome as described in Choo et al. (1989) Science 244:359-362, probes can be derived for detecting the presence of HCV mRNA. HCV RNA can also be detected and quantified, for example, by homogenous reverse transcription polymerase chain reaction (RT-PCR) as described in U.S. Pat. No. 5,527,669, or by nested PCR using a primer set within the 5′ non-coding region of HCV as described, for example, in Kobayashi et al. (1998) J. Gastroenterol. 33:500-507; Tsai et al. (1997) Hepatology 449-458; Di Bischegie et al. (1992) Hepatology 16(3):649-655; and, Garson et al. (1990) Lancet 335:1419-1422. Such methods can be used to identify subjects infected with HCV as well as to monitor the course of HCV infection in a subject to determine whether the subject has an acute or chronic HCV infection.

The term “subject”, as used herein, includes mammals, particularly humans, which can be infected by hepatitis C virus or have HCV-mediated liver disease. Examples of subjects include primates (e.g., humans, and monkeys).

In one embodiment, the invention features methods of identifying HCV-infected subjects likely to be resistant to treatment or to be responsive to treatment for HCV infection. The terms “responsive” and “sustained response” are used interchangeably herein. These terms, as used herein, include treatment or improvement of at least one of the symptoms of HCV infection which results from a treatment for HCV infection. Symptoms of HCV infection include malaise, jaundice, elevated alanine aminotransferase (ALT) levels, the presence of HCV RNA, and the presence of anti-HCV antibodies. Preferably, such treatment or improvement in at least one symptom of HCV infection is maintained over a period of time (e.g., months to years). Subjects demonstrating improvement of a symptom of HCV for at least 6 months are considered responsive to the treatment.

Another embodiment of the invention features methods of identifying HCV-infected subjects unlikely to respond to treatment for HCV infection. The term “resistance”, as used herein, includes HCV-infected subjects unresponsive (“non-responders”) to treatment for HCV infection as defined herein, as well as HCV-infected subjects who suffer a relapse following treatment for HCV infection (“responder-relapsers”). The term “non-responder”, as used herein, includes HCV-infected subjects who fail to demonstrate improvement in at least one symptom, and preferably two or more symptoms of HCV infection in response to treatment for HCV infection. The term “responder-relapser”, as used herein, includes HCV-infected subjects who initially demonstrate improvement of at least one symptom of HCV in response to a treatment, but the improvement is not maintained. Thus, responder-relapsers are HCV-infected subjects who initially demonstrate an improvement in at least one symptom of HCV but revert to pre-treatment symptoms within a period of time (e.g., days, weeks or months) For example, an HCV-infected subject who initially demonstrates decreased serum ALT levels after receiving a treatment for HCV, but over time, e.g., about 6 months after treatment for HCV ceases, demonstrates a return of HCV infection is a responder-relapser. As used herein, the term “relapse” refers to an occurrence of at least one symptom of HCV infection in a subject responsive to HCV treatment. A relapse infection can occur in a subject after a period of about 1 month to about 12 months following treatment for HCV.

The language “treatment for HCV infection” includes a treatment or treatment regimen for HCV infection in a subject. Such treatment can include the use of at least one or a combination of agents which treat or prevent one or more symptoms of HCV in a subject having an HCV infection, e.g., chronic HCV infection. Examples of agents for use in treatment for HCV infection include small molecules, compounds, drugs, proteins, and peptides. Thus, using the methods of the invention, HCV-infected subjects responsive to or unlikely to respond to an agent which is capable of reducing or preventing one or more symptoms of HCV can be identified.

Agents for use in treatment for HCV infection can be incorporated into pharmaceutical compositions suitable for administration. Such compositions typically comprise the agent and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition for use in treatment of HCV is formulated to be compatible with its intended route of administration. Routes of administration for treatment of HCV infection include parenteral, e.g., intravenous, intradermal, subcutaneous and oral administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

In a preferred embodiment of the invention, HCV-infected subjects likely to be responsive to or unlikely to be responsive to interferon treatment for HCV infection are identified. Interferons (IFN) are a family of cytokines which have potent antiviral activity. Interferons for use in treatment of HCV infection include type I interferons. Type I Interferons such as IFN-α and IFN-β also exhibit immunomodulatory activities such as inducing expression of major histocompatibility (MHC) class I and class II proteins, inhibiting the production of IL-1 and stimulating natural killer cells. Interferon α is commercially available from Schering Corporation (Kenilworth, N.J.). Dosage ranges and suitable routes for administration for interferons are known in the art. See Physician's Desk Reference, 51st Edition (Medical Economics Co., Inc., Montvale, N.J., 1997). For example, treatment with IFN-α is usually administered by subcutaneous or intramuscular injections over a 6- to 12 month period at dosages ranging from about 1 mU to 5 mU administered three times a week. See Davis et al. (1994) Am. J. Med. 96 (suppl 1A):41-44. The standard dose of IFN-α treatment is 3 mU three times a week over a 6 month administration period.

Other treatments for HCV infection include treatment with ribavirin (1-β-D-ribofuranosyl-1H-1,2,4-triazole-3carboxamide) or analogs thereof, a nucleoside analog of interferon which also exhibits antiviral activity against RNA viruses. Di Bisceglie et al. (1992) Hepatology 16(3):649-654. Ribavirin is commercially available from Viatek, Incorporated (Costa Mesa, Calif.). Standard dosages and suitable routes of administration for ribavirin are known in the art and described, for example, in Di Bisceglie et al. (1992) Hepatology 16(3):649-654. For example, treatment with ribavirin can be administered orally over a 6 to 12 month period at dosages ranging from about 600 mg/day to about 1,200 mg/day. In one embodiment, such treatments can be used in combination with interferon treatment. Additional treatments for HCV infection include those described in U.S. Pat. No. 5,633,388.

According to the methods of the invention, HCV-infected subjects likely to be responsive to treatment for HCV infection or unlikely to be responsive to treatment for HCV infection are identified by detecting the level of at least one type 2 T helper cell (Th2)-secreted cytokine in the subject during treatment. There are at least two types of T helper subsets: type 1 helper T cells (Th1) which secrete, for example, interleukin (IL)-2 and IFN-γ, and type 2 helper T cells (Th2) which secrete, for example, IL-4, IL-5, IL-6 and IL-10. Th1 cells promote cellular immunity against infectious agents while Th2 cells induce humoral immune responses. The term “Th2 cytokines”, as used herein, includes cytokines secreted by a type 2 helper T cell including IL-4, IL-5, IL-6, and IL-10. Thus, according to the methods of the invention, the level of at least one Th2 cytokine (e.g., IL-4, IL-5, IL-6, and/or IL-10) is detected to determine whether a subject having HCV infection will respond to a treatment for HCV infection. In a preferred embodiment, IL-10 levels are determined to identify HCV-infected subjects responsive to or unlikely to respond to a treatment for HCV, e.g., interferon treatment, e.g., IFN-α treatment.

Methods of detecting the presence of cytokines, e.g., Th2 cytokines, in a sample, are known in the art. Cytokine production can be measured in immune cells either in vitro and/or in vivo. The term “immune cells” as used herein includes hematopoietic cells that produce cytokines, preferably T cells.

For example, in the case of in vitro measurements, immune cells can be removed from a subject and stimulated with antigen (e.g., HCV antigen) in vitro and cytokine production can be measured by determining the cytokine content of the medium in which the cells were grown or can be measured by assaying the transcription of cytokine genes. In vitro stimulation can be performed using a variety of different techniques (e.g., Lohr et al. 1996. Liver. 16:174).

For example, the sample (e.g., a supernatant of cells stimulated in vitro, a serum sample, or a histology sample taken from a liver biopsy) can be contacted with a compound or an agent capable of detecting Th2 cytokines, such that the presence of Th2 cytokines are detected in the biological sample. For example, in the case of samples that comprise whole cells, agents for detecting mRNA of Th2 cytokines include labeled or labelable nucleic acid probes capable of hybridizing to Th2 cytokine mRNA. The nucleic acid probe can be, for example, a nucleotide sequence encoding a Th2 cytokine (e.g., cDNA), or a portion thereof capable of specifically hybridizing under stringent conditions to the Th2 cytokine mRNA. Nucleotide sequences which encode Th2 cytokines, e.g., IL-4, IL-5, IL-6 and IL-10 are known in the art. For example, a nucleotide sequence of the cDNA encoding IL-10 is disclosed in Vieira et al. (1991) Prot. Natl Acad. Sci. USA 88:1172-1176, the contents of which is incorporated by reference. In addition, nucleotide sequences which encode Th2 cytokines are also disclosed, for example, in Norma et al. (1986) Nature 319:640 (IL-4); Lee et al. (1986) Prot. Natl Acad. Sci. USA 83:2061 (IL-4); Tanabe et al. (1987) J. Biol. Chem. 262(34):16580-16584 (IL-5); and, Hirano et al. (1986) Nature 324:73-76 (IL-6), the contents of which are incorporated by reference.

In another embodiment, nucleic acid molecules (e.g., RNA) can be extracted (e.g., from immune cells stimulated in vitro or from a liver sample) using standard techniques. The polymerase chain reaction (PCR) can be used to amplify nucleic acid molecules that encode cytokines using primers that are known in the art and standard techniques, see e.g., Napoli et al. 1996. Hepatology 24:759). PCR can be used on cells that have been stimulated in vitro, (Tsai et al. 1996. Hepatology. 25:449) or can be used to determine the level of cytokine mRNAs in immune cells stimulated in vivo, e.g., taken from a liver biopsy. (Dumoulin et al. 1997. J. Inf. Dis. 175:68).

Additional agents for use in detecting Th2 cytokines include labeled antibodies specific for a Th2 cytokine. Antibodies can be polyclonal, or more preferably, monoclonal. An intact antibody, or a fragment thereof (e.g., Fab or F(ab′)₂) can be used. Standard techniques for antibody production are known in the art. See, e.g., Harlow and Lane, Antibodies, A Laboratory Manual, Cold Spring Harbor Publications, NY (1988). In addition, monoclonal antibodies directed against Th2 cytokines are commercially available. For example, anti-IL-4, anti-IL-5, anti-IL-6, and anti-IL-10 monoclonal antibodies are available from Sigma, St. Louis, Mo. Such antibodies can be used, e.g., in a standard ELISA assay using standard techniques. Quantitative measurements of peripheral Th2 cytokines, can be determined, for example, using cytokine-specific sandwich ELISA for Th2 cytokines, e.g., IL-4 or IL-10, as described in Martinez et al. (1995) Transplantation 59:519-524. In addition, ELISA kits for detecting and quantitating Th2 cytokines in a sample are commercially available. For example, cytokine assays including IL-4 and IL-10 ELISA kits are commercially available from Biosource International, Camarillo, Calif. Such antibodies can also be used, e.g., in an ELISPOT assay (see, e.g., Kobayashi et al. 1998. J. Gastroenterol. 33:500).

The term “labeled”, with regard to a nucleic acid probe or antibody includes direct labeling of the probe or antibody by coupling (i.e., physically linking) a detectable substance to the probe or antibody, as well as indirect labeling of the probe or antibody by reacting it with another reagent that is directly labeled. The label may be coupled directly or indirectly to the antibody or probe by methods known in the art. In addition, a wide variety of labels can be used for detection. Such labels include, for example, radioactive isotopes (e.g., ³H, ¹²⁵I, ³⁵S, ¹⁴C or ³²P), fluorescent compounds (e.g., fluorescein, rhodamine, dansyl or umbelliferone) and chemiluminescent compounds (e.g., luciferin or luminol).

The term “sample” or “biological sample”, as used herein, includes biological fluids (e.g., blood, serum), tissues and cells isolated from a subject, as well as fluids, tissues and cells present within a subject. That is, the detection methods can be used to detect the presence of Th2 cytokine (e.g., Th2 cytokine mRNA) in a biological sample of an HCV-infected subject in vitro as well as in vivo.

In one embodiment, the biological fluid sample used to detect the level of Th2 cytokines is a blood sample. Thus, peripheral Th2 cytokine levels can be determined to identify HCV-infected subjects responsive to or unlikely to respond to a treatment for HCV infection. In vitro techniques which can be used to detect Th2 cytokines in a blood sample include, for example, enzyme linked immunosorbent assays (ELISAs).

In addition, levels of Th2 cytokines can be determined from a biopsy sample. Thus, intrahepatic Th2 cytokine levels can be determined to identify HCV-infected subjects responsive to or unlikely to respond to a treatment for HCV infection. A biopsy sample includes a freeze-dried or fresh frozen section of tissue (e.g., liver tissue) removed from an HCV-infected subject. Methods for detecting Th2 cytokine levels from a biopsy sample include quantitating the expression of Th2 cytokine mRNA. In vitro techniques which can be used to detect mRNA encoding Th2 cytokines include, for example, Northern hybridization. For small biopsy samples reverse transcription polymerase chain reaction (RT-PCR) can be used for detecting Th2 cytokine expression as described, for example, in Fukuda et al. (1995) Clin. Exp. Immunol. 100:446-451. Quantitative measurements of Th2 cytokine levels can then be determined, for example, by competitive PCR with Th2 cytokine specific primers (e.g., IL-4 and/or IL-10 specific primers) as described in Fukuda et al. (1995) Clin. Exp. Immunol. 100:446-451. In addition, dot-blot analysis can be used to quantitate Th2 cytokine levels as described, for example, in Napoli et al. (1996) Hepatology 24(4):759-765.

Determination of the level of at least one Th2 cytokine identifies HCV-infected subjects responsive to or unlikely to respond to treatment for HCV infection. In one embodiment, an elevated level of Th2 cytokines during treatment for HCV indicates that the subject is unlikely to respond to a treatment for HCV. The language “an elevated level of Th2 cytokines” refers to a level of Th2 cytokines in an HCV-infected subject prior to and/or during treatment for HCV as compared to the level of Th2 cytokines in a normal, uninfected subject. For example, in a normal, uninfected subject, Th2 cytokine levels (e.g., IL-10 levels) typically range from undetectable amounts to about 10 pg/ml in serum. HCV-infected subjects unlikely to respond to treatment for HCV infection include subjects determined to have an increased level of at least one Th2 cytokine, e.g., IL-10, compared to normal subjects. In one embodiment, a decrease in the level of a Th2 cytokine during treatment for HCV indicates that an HCV-infected subject is likely to respond to a treatment for HCV. As used herein, the language “a decreased level of Th2 cytokines” refers to a decreased level of Th2 cytokines in an HCV-infected subject as compared to the level of Th2 cytokines in the same HCV-infected subject prior to administration of the treatment. For example, in an HCV-infected subject prior to treatment for HCV infection, Th2 cytokine levels, e.g., IL-10 levels, can be quite variable depending upon the subject, but are often elevated. If, during treatment for HCV infection, if the level of at least one Th2 cytokine, e.g., IL-10, in a subject with HCV infection is decreased when compared with the level in that same subject prior to the initiation of treatment then the HCV-infected subject is liekly to be responsive to treatment for HCV infection. In another embodiment, if the level of at least one Th2 cytokine is comparable to the level observed in normal uninfected subjects, then the HCV-infected subject is likely to be responsive to treatment for HCV infection.

Treatments for HCV are usually administered to a subject over a period of time ranging from several weeks to months. Treatments for HCV infection typically occur over a period from about 2, 3 or 4 months to about 10, 12 or 15 months, preferably from about 6 to 12 months of administration. For example, interferon is typically administered to HCV-infected subjects over a 6 month to 12 month period. However, treatment periods of longer or shorter time periods are also possible.

During a treatment period, the methods of the invention can be used to identify an HCV-infected subject likely to be responsive to a treatment for HCV or resistant to a treatment for HCV. Preferably, Th2 levels are measured prior to treatment or within the first 4 months of treatment for HCV. However, anytime during the treatment for HCV infection, Th2 cytokine levels can be detected to determine whether the treatment is likely to result in a sustained response in the subject.

The present invention is further illustrated by the following examples which in no way should be construed as being further limiting. The contents of all cited references (including literature references, issued patents, published patent applications, and co-pending patent applications) cited throughout this application are hereby expressly incorporated by reference.

### EXAMPLES

### Example I

### Determination of Cytokine Profiles in Hepatitis C Virus-Infected Patients Receiving Interferon Treatment

Serum cytokine levels of IL-6, TNF-α, HGF and IL-10 were measured in forty-five patients with biopsy proven HCV (cirrhosis; n=12) by enzyme immunoassays (ELISA) prior to treatment with interferon-α. Patients were then given interferon-α (INTRON ATM) treatment at a dosage of 3 mU three times a week over a six month period. Three months into therapy with interferon-α serum cytokine levels of IL-6, TNF-α, HGF and IL-10 were measured again. Six months after therapy with interferon-α was terminated, all forty-five patients were classified based on their virological response as non-responders (NR) (n=25), responder-relapsers (RR) (n=12) or sustained responders (SR) (n=8).

TABLE I
Clinical, virological and histological data of patients with
chronic HCV-infection treated with interferon

| | | | Escalated | | | | |
|---|---|---|---|---|---|---|---|
| | | | Dose | | | | |
| RE- | ALT | HIS- | 3 mU→ | IL-6 | TNF | HGF | IL-10 |
| SPONSE | levels | TOLOGY | 5 mU | levels | levels | levels | levels |
| NR | 231 | CAH*/PF | yes | 81 | 3 | 0 | 45 |
| NR | 62 | cah | no | 110 | 9 | 0 | 0 |
| NR | 98 | cah | yes | 100 | 2 | 0 | 0 |
| NR | 113 | CAH | no | 37 | 3 | 0 | 0 |
| NR | 63 | CAH | no | 180 | 217 | 2 | 1022 |
| NR | 103 | CAH/CIR† | yes | 118 | 67 | 2.3 | 340 |
| NR | 110 | CAH | yes | 85 | 58 | 0 | 0 |
| NR | 114 | CAH | yes | 364 | 222 | 3 | 320 |
| NR | 52 | CAH | yes | 129 | 219 | 10 | 1000 |
| NR | 140 | CAH | yes | 128 | 45 | 0 | 0 |
| NR | 25 | CAH | no | 107 | 9 | 2.3 | 140 |
| NR | 75 | CAH | yes | | | | |
| NR | 95 | CAH | no | 38 | 33 | 0 | 0 |
| NR | 130 | CIR | no | 874 | 20 | 14 | 3700 |
| NR | 22 | CAH | yes | 41 | 28 | 0 | 53 |
| NR | | CAH | NA | 35 | 80 | 7.7 | 205 |
| NR | | CAH/CIR | NA | 14 | 38 | 0.5 | 42 |
| NR | | CAH | NA | 8 | 4 | 0 | 1 |
| NR | | CAH | NA | 8 | 13 | 0 | 2 |
| NR | | CAH | NA | 7 | 2 | 0 | 0 |
| NR | | CAH | NA | 508 | 1300 | 18 | 232 |
| NR | | CAH | NA | 7 | 6 | 0 | 0 |
| NR | 40 | INFL | no | 1070 | 1170 | 28 | 2300 |
| RES/REL | 149 | CAH/CIR | yes | 222 | 46 | 12 | 2600 |
| RES/REL | 40 | CAH | no | | | | |
| RES/REL | 87 | CAH/CIR | yes | 116 | 52 | 3 | 270 |
| RES/REL | 142 | CAH | yes | 82 | 39 | 1.4 | 26 |
| RES/REL | 43 | CPH | yes | 22 | 5 | 0 | 0 |
| RES/REL | | CAH | | | | | |
| RES/REL | 57 | CAH | no | 35 | 3 | 0 | 0 |
| RES/REL | 182 | CAH/CIR | no | 39 | 72 | 0 | 0 |
| RES/REL | 70 | CAH | no | 118 | 18 | 3 | 33 |
| RES/REL | 348 | CAH | no | 65 | 229 | 0 | 157 |
| RES/REL | 107 | CAH | no | 172 | 4 | 2.8 | 140 |
| SR | 59 | cal/br | no | 85 | 165 | 1.3 | 25 |
| SR | 82 | CAH | no | 82 | 144 | 4 | 0 |
| SR | | CAH | no | | | | |
| SR | 139 | CAH | no | 85 | 9 | 0 | 0 |
| SR | 150 | CIR | no | 60 | 3 | 0 | 0 |
| SR | | CAH | NA | 8.5 | 9.5 | 0 | 0 |
| SR | | CIR | NA | 8 | 7 | 0 | 0 |
| SR | | CAH | NA | 8 | 2 | 0 | 0 |
| SR | | CAH | NA | 8 | 4 | 0 | 0 |

*CAH refers to Chronic Active Hepatitis; †CIR refers to cirrhosis

Results:

IL-6 and TNF-α were elevated above normal control levels in NR and RR. No significant difference in IL-6 and TNFα levels was seen at baseline or after interferon treatment between the 3 groups (i.e., SR, RR, and NR) and Th1 profile was not predictive of response to interferon. IL-10 was elevated significantly above control in NR and RR but only 1 patient in the SR group had a measurable IL-10 (p<0.01). IL-10 levels after interferon therapy remained non-detectable in SR, fell in NR and RR but did not become undetectable in any patients in which it was elevated. No differences or elevation of HGF was seen in any of the 3 groups. IL-6 and TNFα were elevated in a majority of patients with chronic HCV. Elevated IL-10 at a baseline was seen in 55% of NR, 50% of RR and only 10% of SR and no patients with elevated IL-10 levels responded to interferon alone. These results demonstrate that strong peripheral Th2 response with IL-10 production indicates persistence of viremia and resistance to interferon therapy.

What is claimed:

1. A method for identifying hepatitis C virus-infected subjects responsive to treatment for hepatitis C virus comprising:
determining the level of at least one Th2 cytokine in a subject during treatment for hepatitis C virus infection,
wherein a level of at least one Th2 cytokine is comparable to that measured to an unifected individual indicates that the subject is responsive to treatment for hepatitis C virus.

2. The method of claim 1, wherein the Th2 cytokine is IL-10.

3. A method for identifying hepatitis C virus-infected subjects unlikely to respond to treatment for hepatitis C virus comprising:
determining the level of at least one Th2 cytokine in a subject diagnosed with hepatitis C virus infection,
wherein a level of at least one Th2 cytokine is elevated when compared to that measured in an unifected individual indicates that the subject is unlikely to be responsive treatment for hepatitis C virus.

4. The method of claim 3, wherein the Th2 cytokine is IL-10.

5. The method of claim 1 or claim 3, wherein the level of at least one Th2 cytokine is measured in a peripheral immune cell or in serum.

6. The method of claim 2 or claim 4, wherein the treatment for hepatitis C virus infection is interferon or ribavirin treatment.

7. The method of claim 6, wherein the interferon treatment is type I interferon treatment.

8. The method of claim 7, wherein the interferon treatment is interferon α treatment.

9. The method of claim 1 or claim 3, wherein the subject has a chronic hepatitis C virus infection.

10. The method of claim 1 or claim 3, further comprising obtaining a biological sample from the subject.

11. A method for identifying hepatitis C virus-infected subjects unlikely to respond to treatment with interferon comprising:
a) obtaining a biological sample from the subject diagnosed with hepatitis C virus infection; and
b) determining the level of IL-10 in the sample,
wherein an elevated level of IL-10 indicates that the subject is unlikely to respond to treatment with interferon.

12. A method for identifying hepatitis C virus-infected subjects responsive to interferon treatment comprising:
a) obtaining a biological sample from the subject diagnosed with hepatitis C virus infection; and
b) determining the level of IL-10 in the sample,
wherein a reduction in the pretreatment level of IL-10 indicates that the subject is responsive to interferon treatment.

13. The method of claim 11 or claim 12, wherein the IL-10 is measured in a peripheral immune cell or in serum.

14. The method of claim 13, wherein the interferon treatment is a type I interferon treatment.

15. The method of claim 14, wherein the type I interferon treatment is interferon α treatment.

16. The method of claim 11 or claim 12, wherein the subject has chronic hepatitis C virus infection or HCV-mediated liver disease.
